(19) United States
(12) Patent Application Publication
(10) Pub. No.: US 2012/0108882 A1
(43) Pub. Date: May 3, 2012

(54) Brachytherapy Devices and Related Methods Providing Bioaborbability and/or Asymmetric Irradiation

(76) Inventor: Seth A. Hoedl, Raleigh, NC (US)

(21) Appl. No.: 13/286,624

(22) Filed: Nov. 1, 2011

Related U.S. Application Data
(60) Provisional application No. 61/408,756, filed on Nov. 1, 2010.

(51) Int. Cl. A61M 36/12; A61M 36/04

(52) U.S. Cl. 600/8; 600/3

(57) ABSTRACT

A brachytherapy device includes a sealed housing having a radioactive material therein, and at least one shielding member extending away from the housing and configured to provide radiation shielding on at least one side of the housing.

### RELATED APPLICATIONS

This application claims the benefit of U.S. Provisional Application No. 61/408,756, filed Nov. 1, 2010, the disclosure of which is hereby incorporated by reference in its entirety.

### FIELD OF THE INVENTION

The present invention relates to brachytherapy devices, and in particular, to brachytherapy devices for providing asymmetric irradiation.

### BACKGROUND

The current standard of care for women with early stage breast cancer is breast conserving surgery followed by radiation therapy to the tumor bed. Lumpectomy, or tylectomy, is the surgical removal of a localized tumor from a patient with invasive breast cancer. Historically, radiation treatment has involved external beam radiation delivered to the whole breast, 5 days a week for 5 weeks or more. This time intensive course of therapy has led to poor compliance rates and some 15-30% of women do not start or complete radiation therapy. Further, damage to the heart, lungs, ribs and contralateral breast are all potential consequences of whole breast irradiation. Acute skin toxicity is also a common and uncomfortable side effect of external beam therapy. See Pignol J P, Olivoto I, Rakovitch E, et al. “A multicenter randomized trial of breast intensity-modulated radiation therapy to reduce acute radiation dermatitis.” J Clin Oncol vol. 26, pp. 2085-2092 2008.

To speed radiation treatment delivery and avoid some radiation side effects, high dose rate (HDR) brachytherapy techniques have been developed as adjuvant therapy after breast conserving surgery. HDR brachytherapy techniques for breast treatment involve the temporary placement of catheters or balloons into the breast at the site of the lumpectomy. The catheters or balloons provide an enclosure for a highly radioactive substance, such as 192-Iridium. The radioactivity is typically placed inside the enclosures for one hour twice a day for five days. The enclosures remain in the breast over this period. The enclosures can be intercavity devices such as the Mamosite (Hologic, Bedford, Mass.) or the SAVI Applicator (Cianna, Inc., Aliso Viejo, Calif.), or they can be interstitial devices custom built using standard brachytherapy catheters.

HDR brachytherapy is only appropriate for lumpectomy sites sufficiently deep in the breast so that the skin is spared the intense radiation dose delivered by the HDR source. In addition, because the catheters or balloons remain in the patient for five days and are frequently exposed to the environment outside of the patient for HDR source delivery, there is a non-negligible risk of infection from using the HDR technique. To reduce patient inconvenience and overcome these limitations, Pignol et al. developed a low-dose rate brachytherapy technique that involves the permanent implantation of radioactive 103-Palladium seeds in the breast tissue near the lumpectomy cavity. See: J. P. Pignol M.D., Ph.D., F.R.C.P.C, E. Rakovitch M.D., M.Sc., F.R.C.P.C., B. M. Keller M.Sc., P.A.B.R., R. Sankreacha M.Sc., P.A.B.R. and C. Chartier Ph.D., “Tolerance and Acceptance Results of a Palladium-103 Permanent Breast Seed Implant Phase I/II Study” Intern. J. Rad. Oncology*Biology*Physics, Vol. 73, pp. 1482-1488, April 2009. With this technique, the palladium seeds are implanted in a one hour procedure and permanently remain in the patient. The breast tissue is treated continuously over the lifetime of the radioactive palladium. However, the seeds generally irradiate tissue in all directions around the seeds such that some healthy tissue is also irradiated. Moreover, the seeds remain in the patient even after the palladium is no longer radioactive, and may complicate further imaging of the tissue. Moreover, patients may not wish to have a permanently implanted device.

### SUMMARY

According to some embodiments of the present invention, a brachytherapy device includes a sealed housing having a radioactive material therein, and at least one shielding member extending away from the housing and configured to provide radiation shielding on at least one side of the housing.

In some embodiments, the at least one shielding member is movable between an open position in which the shielding member extends away from the housing and a closed position in which the shielding member is generally adjacent a side of the housing. The housing may be elongated and configured to be implanted in a subject via an implantation needle.

In some embodiments, the housing is configured to be inserted into an implantation needle when the shielding member is in the closed position, and the shielding member is configured to move from the closed position to the open position after implantation in the subject. The at least one shielding member may include at least two shielding members extending in generally opposite directions in the open position. The housing and/or the at least one shielding member in the closed position may have an asymmetrical cross-section indicating a direction of irradiation. The housing may have a triangular cross-section, and the triangular cross-section may define a shielded side of the housing having a shielding layer and two irradiation sides configured to emit radiation from the radioactive material, wherein the two shielding members are attached to opposite ends of the shielding side of the triangular cross-sectional housing. The housing may be configured for implantation via an implantation needle, and the implantation needle may have a corresponding cross-sectional shape that is configured to indicate an implantation direction. In some embodiments, one or more markers are positioned on the implantation needle to indicate an implantation direction.

In some embodiments, the housing comprises a bioabsorbable material. The radioactive material may have an initial radiation activity and the bioabsorbable material may be configured to seal the radioactive material therein for a sealed source time period and to thereafter exhibit sufficient bioabsorption so as to release the radioactive material into the body. The sealed source time period may be at least as long as a duration during which the radioactive material decays to less than about 10% of the initial radiation activity.

In some embodiments, the device includes an imaging marker in the housing. The imaging marker is selected from the group consisting of a gas in an amount sufficient to be detected in an ultrasound image, a ferromagnetic marker in an amount sufficient to be seen on a magnetic resonance image, and a soluble, high density salt in an amount sufficient to be detected on an x-ray image.

In some embodiments, methods of forming a brachytherapy device include forming a sealed housing having a radioactive material therein; forming at least one shielding member extending away from the housing and configured to provide radiation shielding on at least one side of the housing; and providing an implantable medical device from the sealed housing and the at least one shielding member.

In some embodiments, methods of implanting a brachytherapy device are provided. The brachytherapy device includes an elongated, sealed housing having a radioactive material therein and at least one shielding member extending away from the housing and configured to provide radiation shielding on at least one shielded side of the housing. The at least one shielding member is movable between an open position in which the at least one shielding member extends away from the housing and a closed position in which the at least one shielding member is generally adjacent a side of the housing The housing is positioned in an implantation needle when the at least one shielding member is in the closed position. The housing is implanted in a subject using the implantation needle such that, when the housing is implanted in the subject, the at least one shielding member moves from the closed position to the open position to thereby provide a shielded region of tissue and an irradiated region of tissue.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a cross sectional view of a device according to some embodiments of the present invention.

FIG. 2 is a cross sectional view of another device according to some embodiments of the present invention.

FIG. 3 is a top view of a planar device according to some embodiments of the present invention.

FIG. 4 is a cross sectional view of the device of FIG. 3.

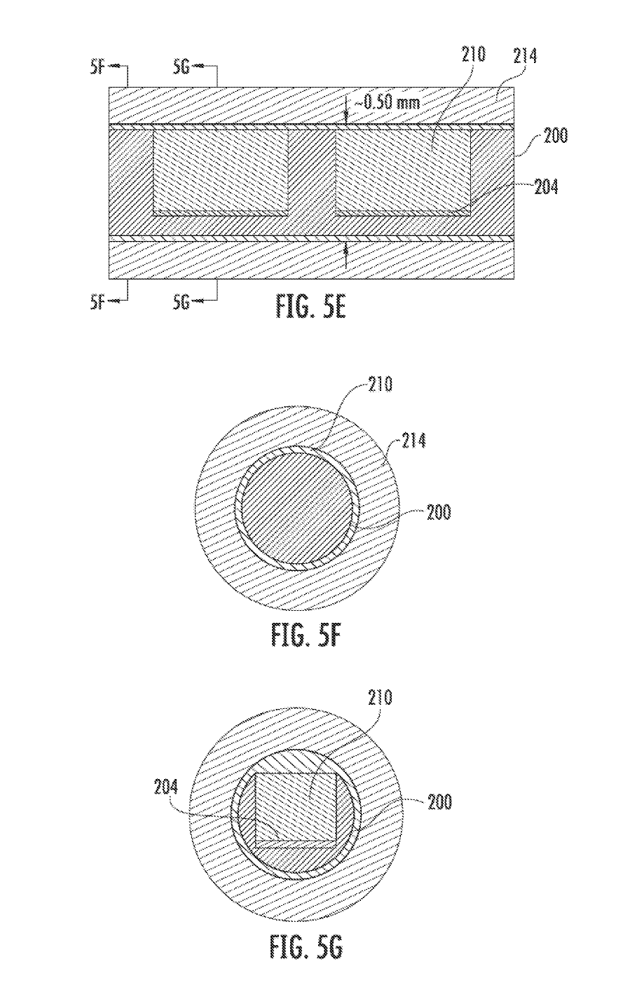

FIG. 5A-5G are cross sectional views of a substrate upon which a radioactive material is deposited according to some embodiments of the present invention.

FIGS. 6 and 7A-7C are cross sectional views of the device of FIGS. 5A-5G that are enclosed in a polymeric tube according to some embodiments of the present invention.

FIGS. 8-9 are cross sectional views of a device having a triangular cross section and shielding wings according to some embodiments of the present invention.

FIG. 10 is a perspective view of a needle and plunger for implanting the device of FIGS. 8-9 according to some embodiments of the present invention.

### DETAILED DESCRIPTION OF EMBODIMENTS OF THE INVENTION

The present invention now will be described hereinafter with reference to the accompanying drawings and examples, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under.” The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present invention. The sequence of operations (or steps) is not limited to the order presented in the claims or figures unless specifically indicated otherwise.

As used herein, the term “globule” refers to a discrete volume of material. Globules of material can be deposited on a planar substrate or in a micro-well on a substrate, for example, using a micro-syringe pump or micro-pipette according to embodiments of the present invention. In some embodiments, the volume of material in globule can be controlled, for example, with an accuracy of better than 10%. Typical sizes of globules are between 5 and 500 nanoliters. In particular embodiments, the globule size is between 30 and 200 nanoliters. In some embodiments, the globules can be spaced apart by about 500-1000 μm.

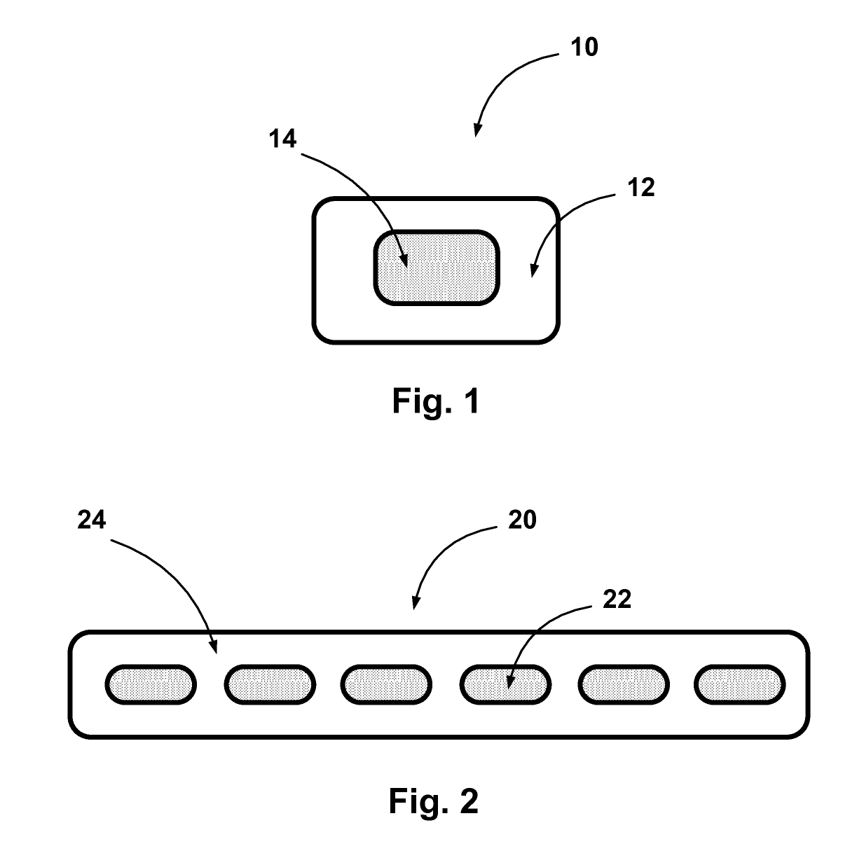

As shown in FIG. 1, a brachytherapy device 10 according to embodiments of the present invention includes a sealed housing or casing 12 and a radioactive material 14. The radioactive material 14 may be a non-soluble radioactive material. The sealed casing 12 may be a biocompatible and/or bioabsorbable material. According to some embodiments, the type of bioabsorbable material in the casing 12 may be evaluated to ensure that the radioactive material 14 is sealed for a sufficient period of time, such as until the radioactive material 14 decays to a safe level, such as less than 10%, less than 5% or even less than 1% of the original radioactivity.

Methods and devices for forming non-soluble radioactive materials are disclosed in U.S. patent application Ser. No. 12/434,131, filed May 1, 2009 and published as U.S. Publication No. 2009/0275793 on Nov. 5, 2009, the disclosure of which is incorporated herein by reference in its entirety.

Any suitable bioabsorbable or biodegradable material may be used, such as copolymers and homopolymers of glycolic acid (GA) and L-lactic acid (LA) or combinations thereof, including copolymers having a blend of these two base materials (e.g., Vicyrl (Polyglactin 910), for instance, is formed with a 90:10 GA-to-LA blend). Another example is a mixture of 18:82 GA-to-LA blend to achieve longer-term stability in the body. Atrisorb, Resolut, or Lactosorb, may also be used.

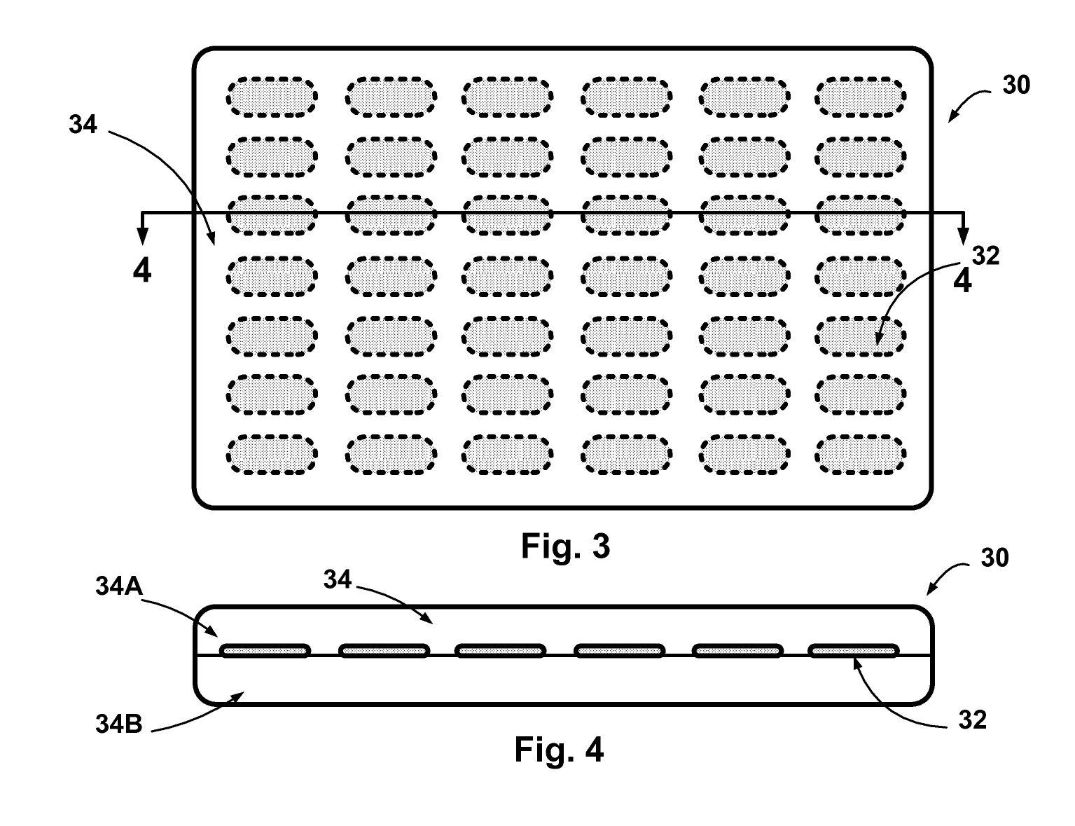

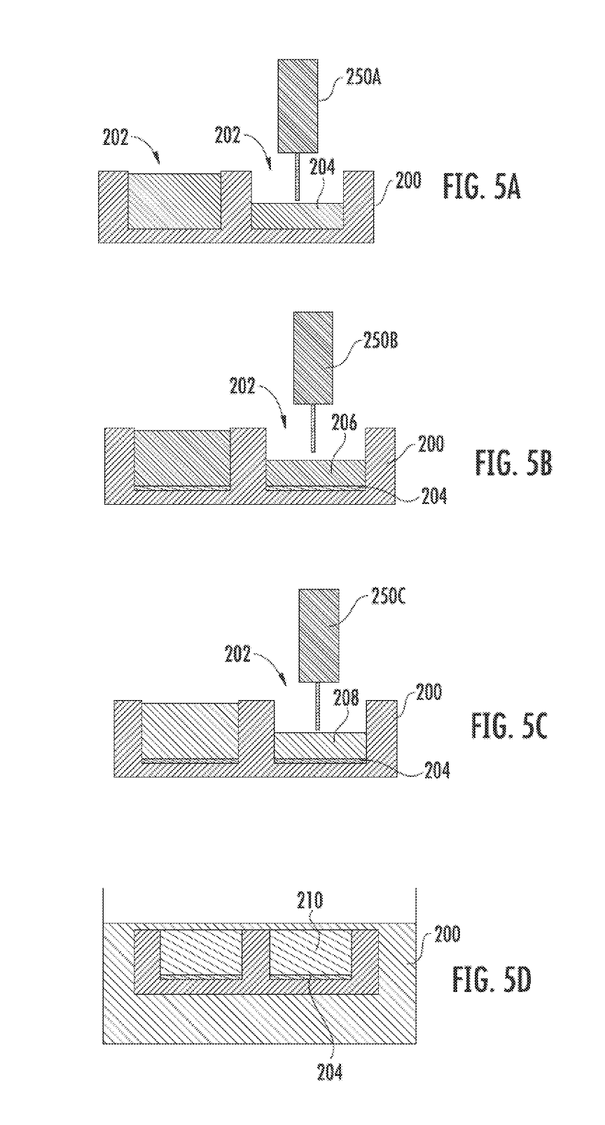

Although the device 10 is illustrated as a “point source” or “seed” shape, any suitable shape of device or encapsulation of the radioactive material may be used. For example, as shown in FIG. 2, a device 20 includes a plurality of globules of radioactive material 22 encapsulated in a sealed casing 24. The linear device 20 may be suitable for implantation, e.g., in breast or prostate tissue. As illustrated in FIG. 3, a planar device 30 includes a radioactive material 32 in a planar sealed casing 34. The sealed casing 34 may include two planar members 34A and 34B with the radioactive material 32 positioned there between.

In some embodiments, one of the planar member 34A and 34B in FIG. 3 includes a radiation shielding material, such as gold. The radiation shielding material may be dispersed throughout one of the planar member 34A and 34B or it may be concentrated or deposited adjacent the radioactive material 32. The radiation shielding material may be useful in applications in which radiation is desired on only one side of the device 30. For example, the device 30 may implanted adjacent a tumor or tumor excision site or other tissue that is identified to be irradiated such as the lung so that one side emits radiation from the radioactive material 32 in a direction toward the tissue that is identified for irradiation and the other side of the device 30 is shielded to protect healthy adjacent tissue.

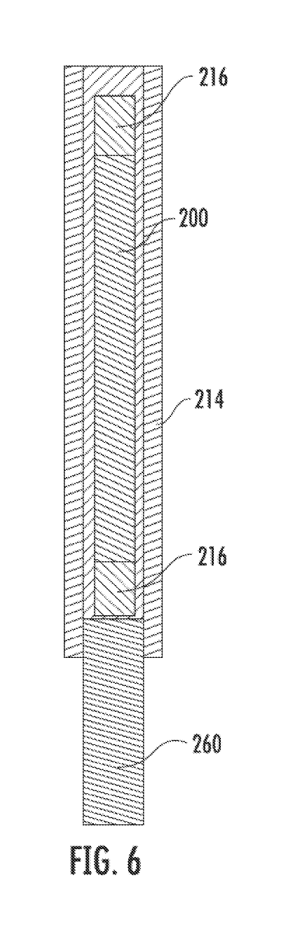

Further embodiments are shown in FIG. 5A-5G. As shown in FIG. 5A, a substrate 200 includes a plurality of microwells 202. A deposition device 250A, such as a micropipette or micro syringe, deposits a radioactive material 204 in the form of a solution in the microwell 202. The volume of the solution of radioactive material 204 can be calculated to match the desired amount of radioactivity in the well 202.

The solution of the radioactive material 204 may be converted into a non-soluble form, for example, by a plasma decomposition process, a chemical decomposition process, and/or a thermal decomposition process as described in U.S. patent application Ser. No. 12/434,131, filed May 1, 2009. For example, as shown in FIG. 5B, a chemical precipitation solution 206 is deposited in the well on the radioactive material 204 by a deposition device 250B, such as an ink jet deposition device. If the solution of the radioactive material 204 is tetraamine palladium chloride in ammonium hydroxide or palladium chloride in hydrochloric acid, the precipitation solution 206 may be a mixture of sodium borohydride and sodium hydroxide in sufficient amounts for a reaction to occur to precipitate out the palladium metal, which is then insoluble. The solution 206 and any remaining amounts of the solution containing the radioactive material 204 may be allowed to dry.

Although the formation of the non-soluble radioactive material 204 is described in FIG. 5B, with respect to the precipitation solution 206, it should be understood that the precipitation solution 206 may be omitted and other techniques may be used to form the non-soluble radioactive material 204. For example, the soluble form of the radioactive material 204 may be a salt of Pd-103, such as tetraamine palladium chloride, which may be dried and then exposed to a plasma treatment (e.g., hydrogen or oxygen plasma) and/or the salt solution may be thermally treated to form non-soluble palladium metal. As described in U.S. patent application Ser. No. 12/434,131, filed May 1, 2009, a plasma treatment may be performed at a pressure of 75-100 mTorr with a power setting of 230 Watts for 2-5 minutes, such as for about 3 minutes. In some embodiments, the radioactive salt solution can be thermally converted to a water-insoluble form at relatively low temperatures. For example, when Pd(NH₃)₄Cl₂ solution dries thoroughly it forms Pd(NH₃)₂Cl₂, which can be thermally decomposed at about 290° C. leaving palladium metal. This processing temperature is consistent with certain polymers, such as silicone, and thus presents a format whereby a polymer can be used as the substrate for the conversion of Pd salt into water insoluble Pd metal.

In FIG. 5C-5D, the wells 202 are filled with a sealant 208, such as medical grade epoxy or resin. Bioabsorbable epoxies, resins or sealants may be used, such as is discussed in U.S. Pat. No. 7,241,846, the disclosure of which is herby incorporated by reference in its entirety. The sealant 208 may then be cured, for example, by thermal or UV curing based on the type of sealant used. The radioactive material 204 is in a water insoluble state and sealed by the sealant 208 to reduce or prevent leakage into the body. As shown in FIGS. 5E-5G, the substrate 200 can also be inserted into a sheath 214 to further reduce the risk of radiation leakage.

As illustrated in FIGS. 5E-5G, the substrate 200 may be inserted into a tube 214. The tube 214 may be formed of a bioabsorbable material, such as Vicyrl, Atrisorb, Resolut or Lactosorb or other suitable material.

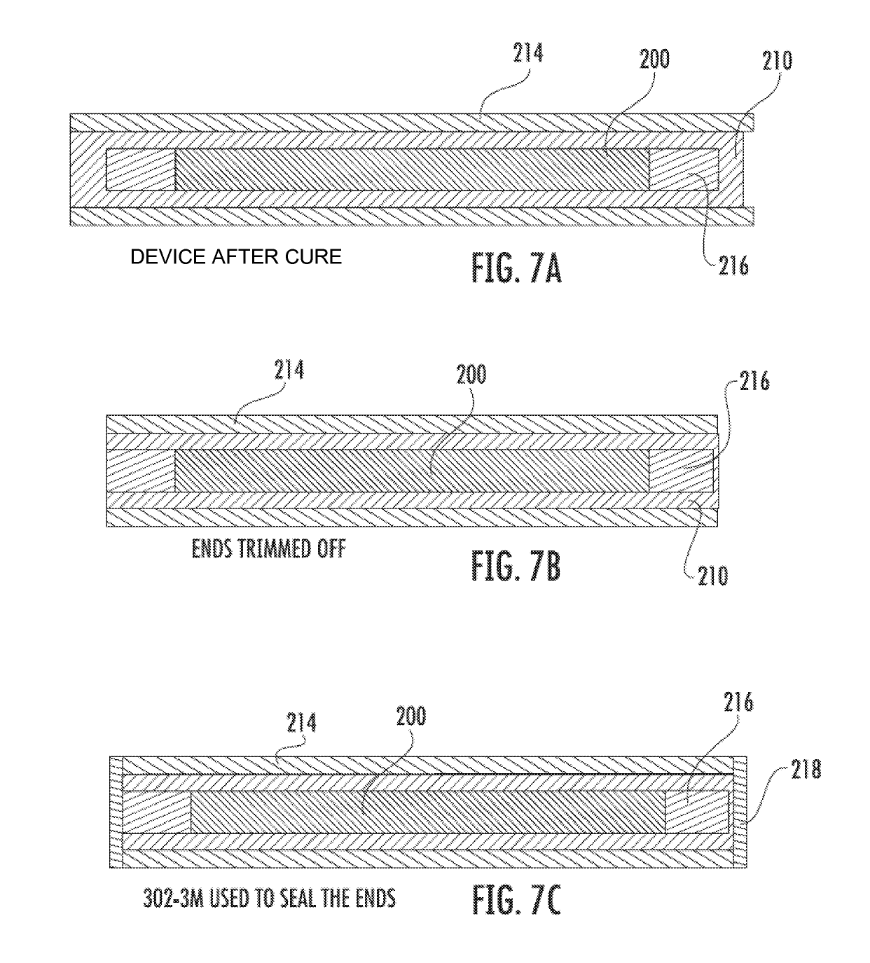

As shown in FIG. 6, an imaging marker 216 may be provided at the ends of the substrate 200 so that the device may be more readily imaged. The imaging marker 216 may be a gas in an amount sufficient to be detected in an ultrasound image or a soluble, high density salt (e.g., potassium iodide) in an amount sufficient to be detected on an x-ray image, or a ferromagnetic substance that can be detected in a magnetic resonance image (MRI). In some embodiments, imaging markers may be placed directly in the wells 202. Any suitable imaging marker may be used, including conventional imaging markers known to those of skill in the art.

The radiographic marker 216 may be configured to allow a sealant 208 to be injected into the sheath 214 by a sealant injector 260. As shown in FIGS. 7A-7B, the ends of the resulting device may be trimmed (FIG. 7B) and a plug 218, such as a polymeric plug, may be inserted on the ends of the device for further sealing and containment of the radioactive material 204.

In some embodiments, some elements or the entire resulting device may be formed of bioabsorbable material, including the substrate 200, the sealant 208, the sheath 214 and/or the plug 218. Moreover, additional markers may be used, and the radiographic marker 216 may be omitted, depending on the desired imaging technique. For example, one of the wells 202 may be a void having a gas therein (e.g., air) that is in an amount sufficient to be detected in an ultrasound image. As another example, very small amounts of ferromagnetic metal may be added to one or more of the wells 202 so that an MRI visibility is enhanced without creating visibility in CT or mammography scans. As yet another example, one or more of the wells may be filled with a soluble, high density salt, such as potassium iodide, that may be visible on x-rays, but also dissolve in bodily fluids when an encapsulating bioabsorbable polymer has broken down.

Although embodiments according to the invention are illustrated with respect to a tubular elongated body as illustrated in FIGS. 2, 5E and 7A-7C, it should be understood that the device could have any suitable non-linear shape. For example, the device could have a curved or bent shape, such as a sinusoidal shape, that reduces migration in the body. The sinusoidal-shaped body may be formed of a flexible shape-memory material such that the sinusoidal-shape may be straightened during implantation, e.g., to fit inside an insertion needle, and become sinusoidal-shaped again after implantation.

Although embodiments according to the invention are illustrated with respect to a tubular body having a cylindrical cross-section as illustrated in FIGS. 5F-5G, it should be understood that any suitable cross-sectional shape may be used.

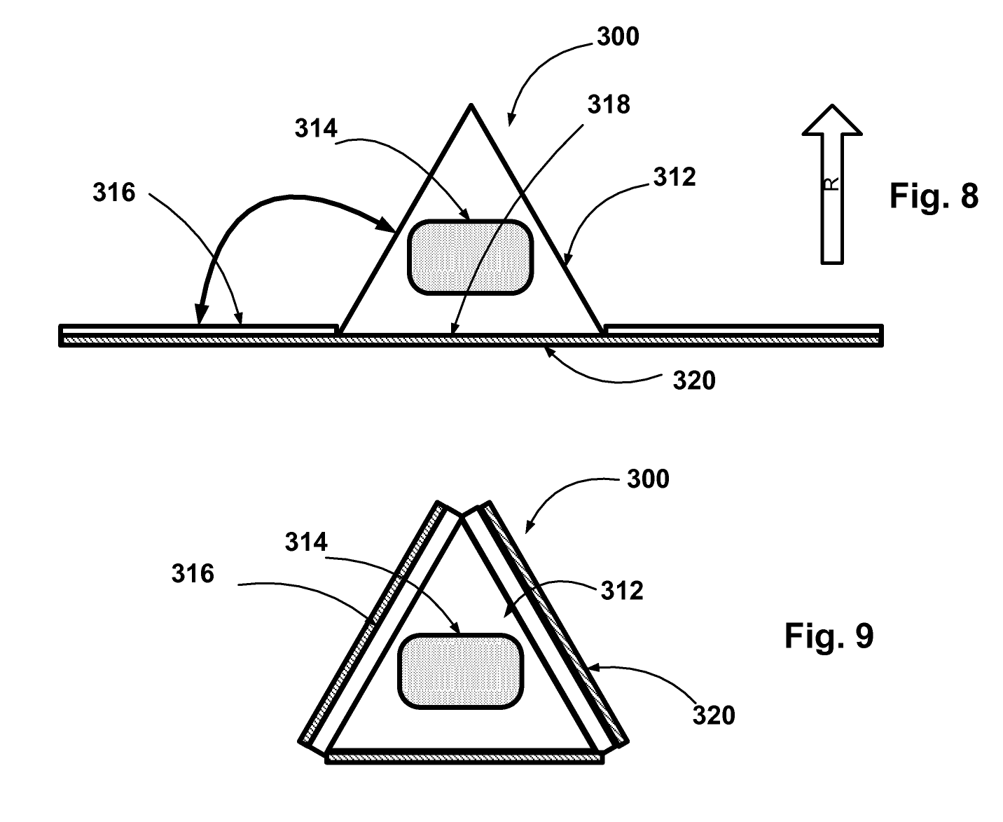

For example, as illustrated in FIGS. 8 and 9, a triangular-shaped cross-sectional device 300 is shown. The device 300 includes a housing or sealed casing 312 and a radioactive material 314. The radioactive material 314 may be deposited as spaced-apart globules along the length of the device 300 as discussed above or the radioactive material 314 may be deposited in a continuous layer. In addition, shielding members or wings 316 may be attached at one end of the sealed casing 312 to provide additional radioactive shielding and to indicate an irradiation direction to a medical professional during an implantation procedure. As illustrated, the wings 316 include a radiation shielding layer 320 that extends along one of the sides 318 of the device. As shown, the shielding layer 320 is a layer on a bottom side of the wings 316 and the side 318; however, it should be understood that a shielding material may be embedded in the device or otherwise attached to the device 300 to provide a radioactive shield that extends generally across one side of the device for controlling a direction of irradiation. Accordingly, asymmetrical irradiation may be provided so that irradiation is increased in some regions around the device 300 (i.e., the direction of arrow R) and reduced in other directions.

Accordingly, the wings 312 and (optionally) a side 318 of the casing 312 adjacent the wings 316 may include a radiation shielding material, such as the layer 320 so that, when the device 300 is implanted, the wings 316 extend to provide additional radiation shielding as shown in FIG. 8. The radiation shielding material may be a gold layer or other shielding material embedded in the side 318 and in the wings 316 or deposited on the outside of the side 318 and wings 316. Radiation from the radioactive material 314 is emitted in the direction indicated by the arrow R. When the device 300 is inserted in tube, such as a needle, for implantation, the wings 316 may be folded against the sides of the sealed casing 312 as shown in FIG. 9. The wings 316 may be formed of a flexible material, such as a flexible polymer, or have a flexible joint portion adjacent the sealed casing 312 to facilitate movement between the open position (after implantation) shown in FIG. 8, and the closed position (when the device 300 is inserted into an implantation tube) as shown in FIG. 9.

In this configuration, the wings 316 provide a visual indication to the medical professional that the radiation is emitted in the direction of arrow R based on the shape of the device 300. Therefore, the medical professional who is implanting the device 300 may orient the device 300 according to a treatment protocol so that the direction of the arrow R faces the tumor or other region identified for irradiation when the wings 316 are folded against the casing 312 for implantation. After implantation, the proper orientation of the device 300 may be verified, e.g., by medical imaging, to determine if the direction of the arrow R is properly facing the region of tissue in which irradiation is desired. Radiographic markers may also be used that may have an asymmetric shape that indicates the direction of the arrow R. For example, the radiographic markers may be cones or pyramids. An array of markers of different sizes located along a line parallel to the arrow R may be used to indicate the direction of the arrow. Markers in the wings 316 and the casing 312 that indicate the direction of the arrow R may also be used. Thus, it should be understood that other cross-sectional shapes and marker placements may be used to indicate the direction of irradiation and/or the orientation of a shielding member with or without using wings. In some embodiments, any asymmetrical cross-sectional shape may be used to visually indicate the direction of irradiation and/or the orientation of a shielding member. Medical imaging markers, including those described herein, may also be used to indicate a direction of irradiation after implantation, for example, by creating a visual indication on a medical image, such as a CT scan, an MRI, or an ultrasound image.

In this configuration, the wings 316 may also serve as anchoring devices to stabilize the device 300 in the patient and reduce or prevent subsequent rotation or motion within the patient's body.

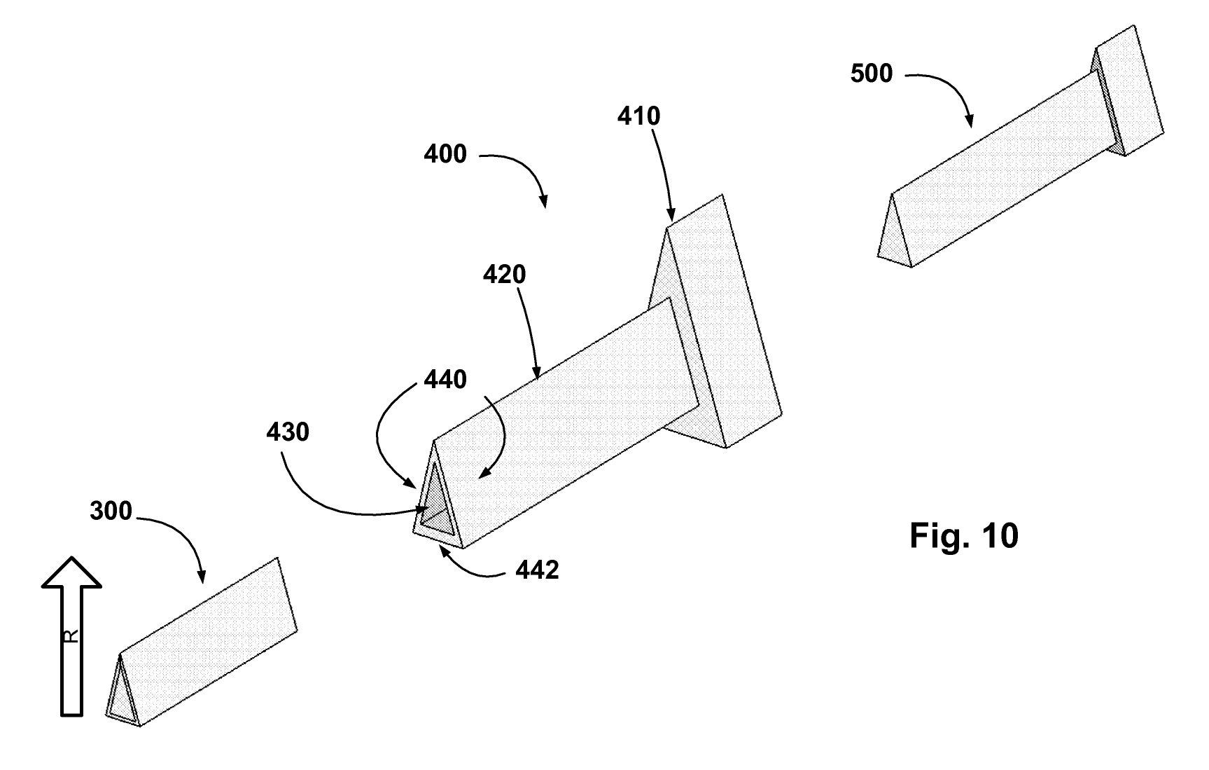

As illustrated in FIG. 10, the device 300 may be implanted using an implantation needle 400 and plunger 500. The needle 400 includes a handle 410 and a needle body 420 having an aperture 430 therethrough. The needle body 420 has a cross-section that is asymmetric with respect to at least one axis. As illustrated, the cross-sectional shape of the needle body 420 is also indicated by the shape of the handle 410 so that the handle 410 indicates a general direction of irradiation to the medical professional. As illustrated, the needle body 420 forms an isosceles triangle with sides 440 of one length and a side 442 of a different length. In this configuration, the device 300 has a corresponding isosceles triangle-shaped cross section that cooperates with the aperture 430 in one orientation. Accordingly, the needle body 420 may be used to define the implantation orientation such that the direction or irradiation (arrow R in FIG. 8) is readily apparent to the medical professional who is implanting the device 300. The device 300 is positioned in the aperture 430, and the needle body 420 is inserted into the body tissue, and the plunger 500 is used to hold the device 300 in the tissue while the needle 400 is removed. The plunger 500 may have a corresponding cross-sectional shape and/or the handle 410 may be labeled and/or have a shape that indicates which direction corresponds to the radioactive side of the device 300 after implantation. Labels and other indicia such as colors may be used to indicate the radioactive side of the device 300, the needle 400, the handle 410 and/or the plunger 500 during implantation.

The needle 400 may be inserted into the patient tissue based on a treatment protocol designed to deliver a given amount of radiation to identified regions of tissue. For example, a plurality of elongated devices 300 may be implanted using a plurality of corresponding needles 400. The needles 400 may be inserted via a standard template, which typically uses round apertures, or a customized template may be used in which the apertures have a size and shape that accepts the needles 400 based on the particular treatment protocol and radiation direction to provide a desired three-dimensional radiation dose.

Wearable radiation shielding may also be provided in particular embodiments of the present invention to reduce radiation exposure to others. For example, an adhesive may be used to adhere a shielding material to the patient's skin in a region adjacent the implanted brachytherapy device. Radiation shielding may be incorporated into clothing or other wearable items, such as items formed from a flexible material. For example, in the case of breast cancer, the patient could wear a bra that is infused with gold or other high density shielding material.

In some embodiments, some elements or the entire resulting device 300 may be formed of bioabsorbable material, including but not limited to the substrate 312, the wings 316 and the shielding 320. In other embodiments, some elements or the entire resulting device 300 may be formed of biocompatible material, including the substrate 312 and the wings 316.

Although embodiments according to the invention are described with respect to the device 300 with a triangular cross-section, it should be understood that any shape may be used. In some embodiments, the shape of the cross-section and/or the position of the wings or shielding member(s) may be used to indicate a shielding side and a radioactive side of the device. For example, a shape that is asymmetric across at least one axis may be used to define the direction of irradiation.

In some embodiments, the radioactive materials described herein may be deposited in spaced-apart globules to control the amount of radiation according to a treatment plan. One or more radioactive elements may be used, including P-32, I-125 and Pd-103. The radioactive material may be deposited as a solution and converted to a non-soluble form and/or the radioactive material may be deposited using globules according to a treatment plan as described in U.S. Pat. No. 7,686,756, filed Aug. 28, 2007 and U.S. application Ser. No. 12,434,131, filed May 1, 2009, the disclosures of which are incorporated herein in their entireties.

In some embodiments, radioactive shielding may be used to provide a generally unidirectional brachytherapy device. In cases of breast cancer, the device could be implanted in such a way so as to expose the tissue adjacent to the lumpectomy but spare the healthy tissue and organs nearby. In addition, such a device may increase the number of patients eligible for LDR brachytherapy for the breast by reducing the anatomical requirements for this form of treatment. For cosmetic reasons, a bioabsorbable version of this device, that is generally absorbed by the patient's body after the radioactive substance has decayed, may be desirable.

Although elongated, shielded devices are illustrated in FIGS. 5-10, other configurations may be used to provide a shielded region and an irradiated region in the tissue after a self-shielded device is implanted. For example, with respect to planar devices used to treat lung cancer, a radiation shielding member on one side of the planar substrate may be used as described herein to provide radiation that is effectively delivered in one direction. A generally unidirectional source may significantly limit the potential for unnecessary exposure to healthy organs or tissues. The availability of a unidirectional, patient specific device may increase the number of patients amenable to the limited surgical approach and decrease the risks associated with the brachytherapy treatment. A bioabsorbable version of the device that is generally absorbed by the patient's body may also be used.

Although embodiments according to the present invention have been discussed with respect to breast and lung cancer, it should be understood that other tumor types may also benefit from a bio-absorbable and/or unidirectional brachytherapy device. For example, a unidirectional device could be placed at different locations in the prostate to maximize dose delivered to diseased tissue while minimizing dose to critical adjacent structures, such as the rectum. It should be understood that other types of cancer may be treated using the methods and devices described herein, including bladder cancer, colon cancer, kidney or renal cancer, skin cancer (melanoma), pancreatic cancer, prostate cancer thyroid cancer, head and neck cancers and soft tissue sarcomas.

In some embodiments, an implantable, LDR brachytherapy device is provided including a non-soluble radioactive material disposed within a sealed casing. The radioactive material has an initial radiation activity and the sealed casing includes a bioabsorbable material configured to seal the radioactive material therein for a sealed source time period and to thereafter exhibit sufficient bioabsorption so as to release the radioactive material into the body. The sealed source time period is at least as long as a duration during which the radioactive material decays to less than about 10% of the initial radiation activity. The device may include a cavity having an imaging marker therein. The imaging marker may be at least one of a ferromagnetic marker in an amount sufficient to be seen on a magnetic resonance image, a gas in an amount sufficient to be detected in an ultrasound image, or a soluble, high density salt in an amount sufficient to be detected on an x-ray image. The device may be further configured according to the various embodiments described herein.

In some embodiments, a method of forming a low-dose-rate (LDR) brachytherapy device includes forming a sealed casing around a radioactive material. The radioactive material has an initial radiation activity and the sealed casing comprising a bioabsorbable material configured to seal the radioactive material therein for a sealed source time period and to thereafter exhibit sufficient bioabsorption so as to release the radioactive material into the body. The sealed source time period is at least as long as a duration during which the radioactive material decays to less than about 10% of the initial radiation activity. A medical device, such as a brachytherapy device, may be formed from the sealed casing and the radioactive material. An imaging marker may be formed in the device, such as a void having gas therein in an amount sufficient to be detected in an ultrasound image, a ferromagnetic marker in an amount sufficient to be seen on a magnetic resonance image and a soluble, high density salt therein in an amount sufficient to be detected on an x-ray image.

The soluble, high density salt may include potassium iodide. The non-soluble radioactive material may include palladium metal.

In some embodiments, a solution comprising a soluble form of the radioactive material is deposited on a substrate, and the soluble form of the radioactive material is converted to a water-insoluble form of the radioactive material on the substrate. The radioactive material may be converted from a soluble form to a water-insoluble form by exposing the substrate to a plasma, heating the substrate so that the radioactive material thermally decomposes, and/or adding a precipitation solution to the soluble form of the radioactive material as described herein. The water-insoluble form of the radioactive material on the substrate may be sealed in the sealed casing, e.g., to form a medical device.

In some embodiments, a solution comprising a soluble form of a radioactive material is deposited by depositing spaced-apart globules of the soluble form of the radioactive material. The globules may a volume of about 30-200 nanoliters.

The sealed casing may be an elongated body. In particular embodiments, the sealed casing includes a plurality of microwells formed in the elongated body, and the elongated body comprises a bioabsorbable polymer. The radioactive material may be deposited in the microwells. The sealed casing may include a bioabsorbable tubular member that is positioned around the elongated body. The sealed casing may include a bioabsorbable resin positioned in the tubular member between the elongated body and the tubular member, which may be cured to form the medical device. The device may be implanted in breast or prostate tissue. In some embodiments, the device may be formed using a planar substrate configured for implantation adjacent lung tissue.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. A brachytherapy device comprising:
a sealed housing having a radioactive material therein;
at least one shielding member extending away from the housing and configured to provide radiation shielding on at least one side of the housing.

2. The brachytherapy device of claim 1, wherein the at least one shielding member is movable between an open position in which the shielding member extends away from the housing and a closed position in which the shielding member is generally adjacent a side of the housing.

3. The brachytherapy device of claim 2, wherein the housing is elongated and is configured to be implanted in a subject via an implantation needle.

4. The brachytherapy device of claim 3, wherein the housing is configured to be inserted into an implantation needle when the shielding member is in the closed position, and the shielding member is configured to move from the closed position to the open position after implantation in the subject.

5. The brachytherapy device of claim 4, wherein the at least one shielding member comprises at least two shielding members extending in generally opposite directions in the open position.

6. The brachytherapy device of claim 5, wherein housing and/or the at least one shielding member in the closed position has an asymmetrical cross-section indicating a direction of irradiation.

7. The brachytherapy device of claim 6, wherein the housing has a triangular cross-section, the triangular cross-section defining a shielded side of the housing having a shielding layer and two irradiation sides configured to emit radiation from the radioactive material, wherein the two shielding members are attached to opposite ends of the shielding side of the triangular cross-sectional housing.

8. The brachytherapy device of claim 7, wherein the housing is configured to be implanted via an implantation needle having a corresponding cross-sectional shape that is further configured to indicate an implantation direction.

9. The brachytherapy device of claim 8, further comprising one or more markers on the implantation needle positioned to indicate an implantation direction.

10. The brachytherapy device of claim 1, wherein the housing comprises a bioabsorbable material.

11. The brachytherapy device of claim 1, a medical imaging marker on the housing having a configuration such that the medical imaging marker indicates a predetermined direction of emitted radiation.

12. The brachytherapy device of claim 11, wherein the medical imaging marker comprises a plurality of medical imaging markers in a shape and/or configuration that indicates the predetermined direction of emitted radiation.

13. The brachytherapy device of claim 11, wherein the medical imaging marker has an asymmetric shape that indicates the predetermined direction of emitted radiation.

14. A method of forming a brachytherapy device comprising:
forming a sealed housing having a radioactive material therein;
forming at least one shielding member extending away from the housing and configured to provide radiation shielding on at least one side of the housing; and
providing an implantable medical device from the sealed housing and the at least one shielding member.

15. The method of claim 14, wherein the at least one shielding member is movable between an open position in which the shielding member extends away from the housing and a closed position in which the shielding member is generally adjacent a side of the housing.

16. A method of implanting a brachytherapy device, the brachytherapy device comprising an elongated, sealed housing having a radioactive material therein and at least one shielding member extending away from the housing and configured to provide radiation shielding on at least one side of the housing, wherein the at least one shielding member is movable between an open position in which the at least one shielding member extends away from the housing and a closed position in which the at least one shielding member is generally adjacent a side of the housing, the method comprising:
positioning the housing in an implantation needle when the at least one shielding member is in the closed position; and
implanting the housing in a subject using the implantation needle such that, when the housing is implanted in the subject, the at least one shielding member moves from the closed position to the open position to thereby provide a shielded region of tissue and an irradiated region of tissue.

17. The method of claim 16, wherein the at least one shielding member comprises at least two shielding members extending in generally opposite directions in the open position.

18. The method of claim 16, wherein the housing and/or the at least one shielding member in the closed position has an asymmetrical cross-section indicating a direction of irradiation, wherein implanting the housing in a subject using the implantation needle comprises orienting the housing and the implantation needle in a direction such that, after implantation, the direction of irradiation is facing a desired irradiation location in the subject.

19. The method of claim 16, wherein the housing has a triangular cross-section, the triangular cross-section defining a shielded side of the housing having a shielding layer and two irradiation sides configured to emit radiation from the radioactive material, wherein the two shielding members are attached to opposite ends of the shielding side of the triangular cross-sectional housing, and orienting the housing and the implantation needle comprises orienting the housing and the implantation need such that the two irradiation sides are configured to emit radiation to the desired irradiation location in the subject.

20. The method of claim 19, wherein the housing and/or the implantation needle further comprise one or more markers indicating an implantation direction, the method comprising determining an orientation of the housing and/or the implantation needle based on the one or more markers.

21. The method of claim 16, wherein the housing comprises a bioabsorbable material.

22. The method of claim 21, wherein the device includes a medical imaging marker on the housing and having a configuration such that the medical imaging marker indicates a predetermined direction of emitted radiation.

23. The brachytherapy device of claim 22, wherein the medical imaging marker comprises a plurality of medical imaging markers in a shape and/or configuration that indicates the predetermined direction of emitted radiation.

24. The brachytherapy device of claim 22, wherein the medical imaging marker has an asymmetric shape that indicates the predetermined direction of emitted radiation.

25. The method of claim 16, further comprising positioning a wearable, flexible material on a subject in which the device is implanted, the material comprising a shielding layer.

26. A brachytherapy device comprising:
a sealed housing having a radioactive material therein;
at least one shielding member on the housing and configured to provide radiation shielding on at least one side of the housing such that radiation is emitted in a predetermined direction; and
a medical imaging marker on the housing and having a configuration such that the medical imaging marker indicates the predetermined direction of emitted radiation.

27. The brachytherapy device of claim 26, wherein the medical imaging marker comprises a plurality of medical imaging markers in a shape and/or configuration that indicates the predetermined direction of emitted radiation.

28. The brachytherapy device of claim 26, wherein the medical imaging marker has an asymmetric shape that indicates the predetermined direction of emitted radiation.
